(19) United States
(12) Patent Application Publication
(10) Pub. No.: US 2026/0335120 A1
(43) Pub. Date: Oct. 29, 2026

(54) URINE COLLECTION ASSEMBLY INCLUDING A POROUS MATERIAL AND METHOD TO MANUFACTURE THE URINE COLLECTION ASSEMBLY

(71) Applicant: PUREWICK CORPORATION, Covington, GA (US)

(72) Inventors: Zhihui Yin, Lilburn, GA (US); Kamil Szymaniak, Dacula, GA (US); Michael Anderson, Monroe, GA (US)

(21) Appl. No.: 19/110,938

(22) PCT Filed: Sep. 16, 2022
(86) PCT No.: PCT/US2022/043818

§ 371 Date: Mar. 12, 2025

(51) Int. Cl. A61F 5/455

(52) U.S. Cl. CPC A61F 5/455

(57) ABSTRACT

Embodiments are directed to urine collection assemblies including a fluid impermeable barrier at least defining a chamber, at least one opening, and a fluid outlet and a porous material disposed in the chamber. The porous material exhibits a cylindrical shape disposed in the chamber and includes a porous support material and a hydrophilic cover sheet disposed over the porous support material. A seal is applied along an entire intersection plane of the porous material, the seal configured to hold the porous material in a cylindrical shape. The urine collection assembly further includes a tube. The tube is disposed within the porous material and the tube is in fluid communication with the fluid outlet. Embodiments are also directed towards methods of forming urine collection assemblies.

### BACKGROUND

A person may have limited or impaired mobility so typical urination processes are challenging or impossible. For example, a person may experience or have a disability that impairs mobility. A person may have restricted travel conditions such as those experienced by pilots, drivers, and workers in hazardous areas. Additionally, sometimes bodily fluids collection is needed for monitoring purposes or clinical testing.

Urinary catheters, such as a Foley catheter, can address some of these circumstances, such as incontinence. Unfortunately, urinary catheters can be uncomfortable, painful, and can lead to complications, such as infections. Additionally, bed pans, which are receptacles used for the toileting of bedridden individuals are sometimes used. However, bedpans can be prone to discomfort, spills, and other hygiene issues.

### SUMMARY

Embodiments are directed to urine collection assemblies and methods of forming a urine collection assembly. In an embodiment, a method of forming a urine collection assembly can include cutting a tube and a porous material. The porous material can include a porous support material and a hydrophilic cover sheet disposed over the porous support material. The method can further include wrapping the porous support material and the hydrophilic cover around a gripping tool to form a cylinder. In an embodiment, the gripping tool can include a pin extending from an outer surface of the gripping tool. The method can also include withdrawing the gripping tool from the wrapped porous material to form a channel within the porous material. In an embodiment the method further includes inserting the tube into the channel to form a sub-assembly and positioning the sub-assembly into a fluid impermeable barrier.

In an embodiment, a method of forming a urine collection assembly is disclosed. The method can include forming a porous material, the porous material including a first edge and a second edge opposite the first edge. The method can further include shaping the porous material into a cylinder, the cylinder defining a channel extending through a centerline of the porous material. The method can also include sealing the first edge of the porous material to the second edge of the porous material. In an embodiment, the method further includes inserting a tube into the channel to form a sub-assembly and positioning the sub-assembly into a fluid impermeable barrier.

In an embodiment, a urine collection assembly is disclosed. The urine collection assembly includes a fluid impermeable barrier at least defining a chamber, at least one opening, and a fluid outlet. The urine collection assembly also includes a porous material formed into a cylindrical shape disposed in the chamber. The porous material includes a porous support material, a hydrophilic cover sheet disposed over the porous support material, and a seal applied along an entire intersection plane of the porous material, the seal configured to hold the porous material in a cylindrical shape. The urine collection assembly also includes a tube. The tube is disposed in the porous material and the tube can be in fluid communication with the fluid outlet.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1A is an isometric view of a urine collection assembly including a porous material, according to an embodiment.

FIG. 1B is a cross-sectional schematic of the urine collection assembly shown in FIG. 3A taken along plane B-B.

FIG. 1C is a cross-sectional schematic of the urine collection assembly shown in FIG. 3A taken along plane a-a.

FIG. 1D is a side elevation view of a sub-assembly of a urine collection assembly, according to an embodiment.

FIG. 2 is a block diagram of a urine collection system including a urine collection assembly, according to an embodiment.

FIG. 3 is a flow diagram of a method of forming a urine collection assembly, according to an embodiment.

FIG. 4 is a flow diagram of a method of forming a urine collection assembly, according to an embodiment.

FIG. 5A is an isometric view of a gripping tool including a series of pins, according to an embodiment.

FIG. 5B a cross-sectional schematic of a gripping tool, according to an embodiment.

### DETAILED DESCRIPTION

Embodiments are directed to urine collection assemblies and methods of forming the urine collection assemblies. In some examples, a urine collection assembly may include a fluid impermeable barrier at least defining a chamber, at least one opening, and a fluid outlet. A porous material may be disposed in the chamber. The porous material can include a porous support material and a hydrophilic cover sheet disposed over the porous support material. The urine collection assembly may further include a tube. The porous material can be configured to surround the tube and the tube may be in fluid communication with the fluid outlet. Embodiments are also directed towards urine collection systems including such urine collection assemblies.

During use, the fluid collection assembly may be positioned such that the hydrophilic cover sheet is positioned adjacent to and contacts a urethral opening (e.g., vaginal) of a female. The female may discharge one or more bodily fluids (e.g., urine). The discharged urine may be received into the porous material. The urine may flow through the porous material to an inlet of the tube positioned through the fluid outlet defined by the fluid impermeable barrier. The urine then may flow through the tube as it is removed from the fluid collection assembly. In an embodiment, a vacuum may be provided from the tube to draw the urine into and through the porous material to the inlet of the tube. The vacuum may also facilitate flowing the urine through the tube. The vacuum may be provided from a vacuum source that is in fluid communication with the tube.

Conventional fluid collection assemblies may include a porous material positioned to initially receive bodily fluids from the individual using such conventional fluid collection assemblies. Such porous materials are hydrophobic or otherwise configured to wick bodily fluids into the conventional fluid collection assemblies. However, many of the conventional porous materials positioned to initially receive the bodily fluids from the individual can be ineffective at capturing the urine discharged from the individual, which increases the likelihood that bodily fluids leak from the fluid collection assemblies. Further, it has been found that many of the conventional porous materials remain wet after the individual discharges bodily fluids which prevents the conventional fluid collection assemblies from being used for a prolonged period of time (e.g., periods of time greater than 12 hours) without causing skin degradation of the individual, which can be a great discomfort.

The urine collection assembly and the porous material disposed therein can remedy at least some of these issues associated with the porous materials of conventional fluid collection assemblies. For example, the hydrophilic cover sheet is configured to form a sealed layer when the hydrophilic cover sheet becomes wetted to promote vacuum function within the assembly. The porous support material can efficiently receive the bodily fluids, thereby the porous material may prevent or at least inhibit leaking of the bodily fluids. The hydrophilic cover sheet may also be configured to dry quicker than the conventional porous materials after receiving the bodily fluids, which allows the urine collection assembly to be used for prolonged periods of time (e.g., periods of time greater than about 24 hours, such as about 24 hours to about 36 hours, about 30 hours to about 42 hours, or about 36 hours to about 48 hours). The hydrophilic cover sheet may efficiently receive the bodily fluids and remain dry, for example, due to at least one or more of the hydrophilicity of the material that includes the cover sheet or the properties of the porous material, as discussed in more detail below.

The porous support material promotes flow of the urine towards an inlet of the tube, which removes the urine from the urine collection assembly. The porous support material can include a 3D structure configured to promote such fluid flow, for example, due to at least one or more of the hydrophobicity of the porous support material, the thickness, or the surface density, as discussed in more detail below.

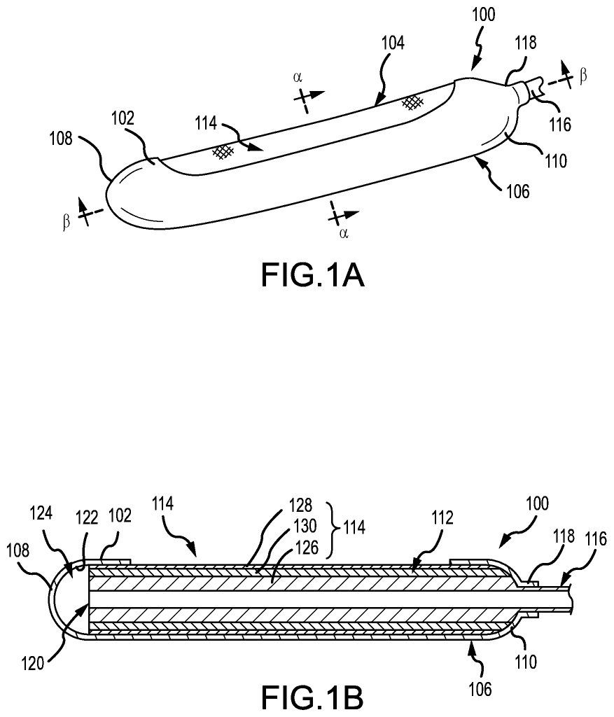

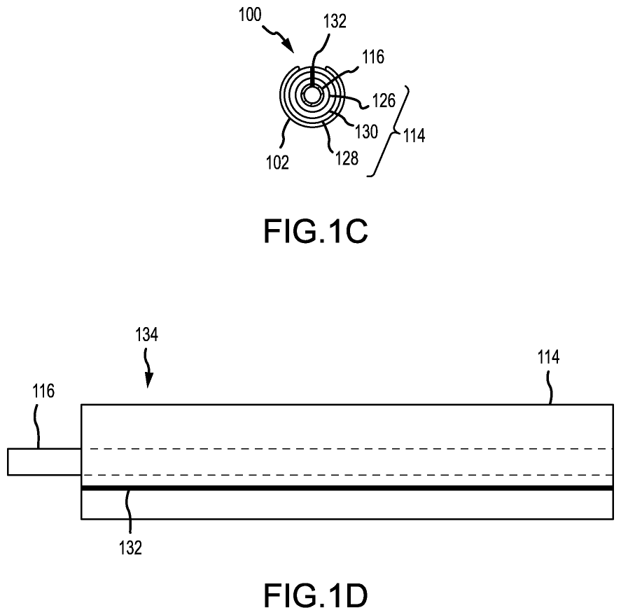

FIG. 1A is an isometric view of a urine collection assembly 100, according to an embodiment. FIG. 1B is a cross-sectional schematic of the urine collection assembly 100 taken along plane β-β, shown in FIG. 1A. The urine collection assembly 100 is an example of a female urine collection assembly for receiving and collecting bodily fluids (e.g., urine) from a female. The urine collection assembly 100 includes a fluid impermeable barrier 102. The fluid impermeable barrier includes an opening 104 defined by a fluid impermeable outer surface 106. The fluid impermeable barrier 102 may be formed of any suitable fluid impermeable material(s), such as a fluid impermeable polymer (e.g., silicone, polypropylene, polyethylene, polyethylene terephthalate, neoprene, a polycarbonate, etc.), natural rubber, another suitable material, any other fluid impermeable material disclosed herein, or combinations thereof. As such, the fluid impermeable barrier 102 substantially prevents the bodily fluids from passing through the fluid impermeable barrier 102. In an example, the fluid impermeable barrier 102 may be air permeable and fluid impermeable. At least one or more portions of the outer surface 106 of the fluid impermeable barrier 102 may be formed from a soft and/or smooth material, thereby reducing chaffing.

In some examples, the fluid impermeable barrier 102 may be tubular shaped, such as substantially cylindrical (as shown); or oblong, prismatic, or flattened tubes. In some examples, during use, the outer surface 106 of the fluid impermeable barrier 102 may contact the individual. The fluid impermeable barrier 102 may be sized and shaped to fit between the labia and/or the gluteal cleft between the legs of a female user.

The opening 104 may provide an ingress for urine to enter the urine collection assembly 100. The opening 104 may be defined by the fluid impermeable barrier 102 such as by an inner edge of the fluid impermeable barrier 102, thereby enabling urine to enter the urine collection assembly 102 through the opening 104.

The opening 104 may be an elongated hole in the fluid impermeable barrier 102. For example, the opening 104 may be defined as a cut-out in the fluid impermeable barrier 102 extending from a distal end region 108 to a proximal end region 110. The opening 104 may be located and shaped to be positioned adjacent to a female urethral opening. The opening 104 may have an elongated shape because the space between the legs of a female is relatively small when the legs of the female are closed, thereby only permitting the flow of the bodily fluids along a path that corresponds to the elongated shape of the opening 104 (e.g., longitudinally extending opening 104).

The fluid impermeable barrier 102 at least partially defines a chamber 112. For example, the interior surface(s) 122 of the fluid impermeable barrier 102 at least partially defines the chamber 112 within the urine collection assembly 100. The fluid impermeable barrier 102 temporarily stores the bodily fluids in the chamber 112.The urine collection assembly 100 also includes the porous material 114 disposed in the chamber 112. The urine collection assembly 100 may be positioned proximate to the female urethral opening and the urine may enter the chamber 112 of the urine collection assembly 100 via the opening 104. The urine collection assembly 100 is configured to receive the bodily fluids into the chamber 112 via the opening 104. When in use, the opening 104 may have an elongated shape that extends from a first location below the urethral opening to a second location above the urethral opening.

The urine collection assembly 100 may further include a tube 116 disposed through a fluid outlet 118 defined by the fluid impermeable barrier 102, such that an inlet 120 of the tube 116 is disposed in the chamber 112. In some examples, the fluid impermeable barrier 102 may define the fluid outlet 116 sized to receive the tube 116. The tube 116 may be disposed in the chamber 112 via the fluid outlet 118. The fluid outlet 118 may be sized and shaped to form an at least substantially fluid tight seal against the tube 116 thereby substantially preventing the bodily fluids from escaping the chamber 112.

As previously discussed, the porous material 114 is disposed in the chamber 112. The porous material 114 may be formed into and exhibit a generally cylindrical shape. In an embodiment, the porous material 114 may be provided in a sheet. In such an embodiment, the porous material 114 may be rolled into a generally cylindrical shape with opposing edges thereof contacting each other as discussed further in methods described below.

In some examples, the porous material 114 may be cut to a length that at least substantially completely fill the portions of the chamber 112 that are not occupied by the tube 116. In some examples, the porous material 114 may not substantially completely fill the portions of the chamber 112 that are not occupied by the tube 116. In such an example, the urine collection assembly 100 includes a reservoir 124 or sump disposed in the chamber 112.

The reservoir 124 or sump may be a substantially unoccupied portion of the chamber 112. The reservoir 124 may be defined between the fluid impermeable barrier 102 and the porous material 114. The urine in the chamber 112 may flow through the porous material 114 to the reservoir 124. The reservoir 124 or sump may retain of the bodily fluids therein. While depicted in the distal end region 108, the reservoir 124 or sump may be located in any portion of the chamber 112 such as the proximal end region 110. The reservoir 124 or sump may be located in a portion of the chamber 112 that is designed to be located in a gravimetrically low point of the urine collection assembly 100 when the urine collection assembly 100 is in use.

In some examples, the porous material 114 may include a porous support material 126 and a hydrophilic cover sheet 128 disposed over the porous support material 126. The porous material 114 may be disposed in the chamber 112 such that the hydrophilic cover sheet 128 is positioned closer to the urethral opening of the individual than the porous support material 126. For example, the hydrophilic cover sheet 128 may extend across the opening 104 and be exposed to an exterior of the urine collection assembly 100. As such, the hydrophilic cover sheet 128 may contact the vaginal region of an individual when the urine collection assembly 100 is positioned adjacent to a vaginal region. The porous material 114 may also be positioned such that the porous support material 126 defines a bore that is configured to receive the tube 116, when the urine collection assembly 100 is assembled.

The tube 116 may be at least partially disposed in the chamber 112. The tube 116 may be used to remove urine from the chamber 112. The tube 116 includes at least one wall defining an inlet 120, an outlet (not shown) downstream from the inlet 120, and a passageway. The outlet of the tube may be operably coupled to a vacuum source, such as a vacuum pump for withdrawing fluid from the chamber 112 through the tube 116. For example, the tube 116 may extend into the fluid impermeable barrier 102 from the proximal end region 110 and may extend to the distal end region 108 to a point proximate to the reservoir 124 therein such that the inlet 120 is in fluid communication with the reservoir 124. The tube 116 fluidly couples the chamber 112 with a fluid storage container (not shown) or the vacuum source (not shown).

The porous material 114 may surround the tube 116. In an embodiment, the tube 116 extends from the fluid outlet 118, through the porous material 114, to a location that is proximate to the reservoir 124 and near the distal end region 108. In such an embodiment, the inlet 120 may not extend into the reservoir 124 and, instead, the inlet 120 may be disposed within the porous material 114 or at a terminal end thereof. In an embodiment, the tube 116 is at least partially disposed in the reservoir 124 and the inlet 120 may be extended into or be positioned in the reservoir 124. Thus, the inlet 120 of the tube 116 may be aft of the reservoir 124 or sump, flush with the porous material 114, or extending into the reservoir 124. The urine collected within the urine collection assembly 100 may be removed from the chamber 112 via the tube 116.

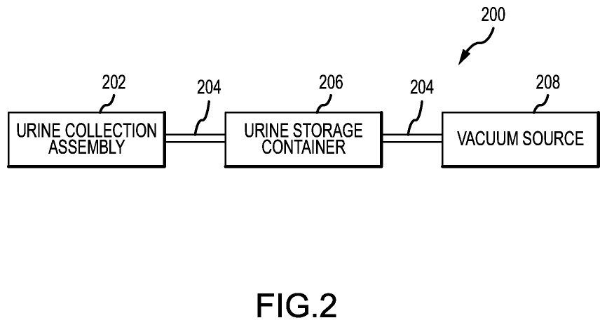

Locating the inlet 120 at or near a location expected to be the gravimetrically low point of the chamber 112 when worn by an individual enables the tube 116 to receive more of the urine than if tube inlet 120 was located elsewhere and reduce the likelihood of pooling (e.g., pooling of the urine may cause microbe growth and foul odors). The tube inlet 120 and the outlet of the tube 116 are configured to fluidly couple (e.g., directly or indirectly) the vacuum source (not shown) to the chamber 112 (e.g., the reservoir 124). As the vacuum source (shown in FIG. 2) applies a vacuum/suction in the tube 116, the urine in the chamber 112 (e.g., at the distal end region 108 such as in the reservoir 124) may be drawn into the inlet 120 and out of the urine collection assembly 100 via the tube 116.

In an example, the hydrophilic cover sheet 128 may contact the anatomical area directly and receive the urine. The hydrophilic cover sheet 128 can pull the urine into the porous material 114, thereby allowing the hydrophilic cover sheet 128 to efficiently receive the bodily fluids. Also, the hydrophilic cover sheet 128 distributes the urine through the hydrophilic cover sheet 128, which allows the hydrophilic cover sheet 128 to receive a large quantity in a relatively short period of time and facilitate transferring the urine from the hydrophilic cover sheet 128 to the porous support material 126. The hydrophilic cover sheet 128 can include a non-woven structure. In some examples, the hydrophilic cover sheet 128 can include a fabric gauze.

The hydrophilic cover sheet 128 may be selected to exhibit a surface density of between about 10 g/m² to about 200 g/m². In some examples, the surface density of the hydrophilic cover sheet 128 may be between about 75 g/m² to about 125 g/m², about 100 g/m² to about 150 g/m², about 125 g/m² to about 175 g/m², or about 150 g/m² to about 200 g/m². The surface density of the hydrophilic cover sheet 128 is a function of the density and thickness of the hydrophilic cover sheet 128. As such, the surface density of the hydrophilic cover sheet 128 may be selected for any of the same reasons as the thickness of the hydrophilic cover sheet 128.

The hydrophilic cover sheet 128 may exhibit a thickness that is significantly less than a thickness of the porous support material 126. For example, the thickness of the hydrophilic cover sheet 128 may be about 400 μm or less, such as about 350 μm or less, about 300 μm or less, about 250 μm or less, about 200 μm or less, about 150 μm or less, about 100 μm or less, or in ranges of about 50 μm to about 150 μm, about 100 μm to about 200 μm, about 150 μm to about 250 μm, about 200 μm to about 300 μm, or about 250 μm to about 400 μm. The thickness of the hydrophilic cover sheet 128 may allow the hydrophilic cover sheet 128 to efficiently receive the urine since the distance that the urine need to flow through the hydrophilic cover sheet 128 is reduced. The thickness of the hydrophilic cover sheet 128 may also allow the hydrophilic cover sheet 128 to quickly dry since the thickness of the hydrophilic cover sheet 128 only allows the hydrophilic cover sheet 128 to hold a relatively small quantity of urine at any given time. The relatively small quantity of urine present in the hydrophilic cover sheet 128 may be easily removed (e.g., evaporated) into the atmosphere or by air flow caused by a vacuum applied to the chamber 112 of the fluid collection assembly 100. Conventional selection of materials for fluid collection assemblies avoids using hydrophilic materials, especially in portions proximate to the urethral opening, since hydrophilic materials tend to retain the urine and remain wet. As such, conventional selection of materials for fluid collection assemblies tend to use hydrophobic materials since hydrophobic materials do not retain large quantities of fluid. However, hydrophobic materials may not effectively receive bodily fluids such as urine.

It is noted that, generally, decreasing the thickness of the hydrophilic cover sheet 128 increases the efficiency at which the hydrophilic cover sheet 128 receives the bodily fluids and increases how quickly the hydrophilic cover sheet 128 may dry. However, decreasing the thickness of hydrophilic cover sheet 128 also decreases the durability of the hydrophilic cover sheet 128 and may also limit diffusion of the urine into the hydrophilic cover sheet 128 in a direction that is generally parallel to a longitudinal axis β-β which, in turn, may facilitate flow of the urine from the hydrophilic cover sheet 128 into the porous support material 126.

As previously discussed, the porous material 114 includes the porous support material 126. In an example, the porous support material 126 is a woven fabric. In some examples, the porous support material 126 can include at least one of polyester, polypropylene, polyethylene, nylon, spun nylon fibers, vertically woven bamboo, cotton, or cellulose. Further examples of fluid collection assemblies that may include the porous materials disclosed herein are disclosed in U.S. patent application Ser. No. 15/612,325 filed on Jun. 2, 2017, U.S. patent application Ser. No. 15/260,103 filed on Sep. 8, 2016, U.S. Pat. No. 10,390,989 filed on Sep. 8, 2016, U.S. Provisional Patent Application No. 63/067,542 filed on Aug. 19, 2020, and U.S. patent application Ser. No. 16/433,773 filed on Jun. 6, 2019, the disclosures of each of which are incorporated herein, in its entirety, by this reference.

The porous support material 126 may exhibit a thickness that is about 20 mm or less, such as about 15 mm or less, about 10 mm or less, about 5 mm or less, about 2 mm or less, or in ranges of about 2 mm to about 5 mm, about 5 mm to about 10 mm, about 10 mm to about 15 mm, or about 15 mm to about 20 mm. The porous support material 126 may be hydrophobic in some embodiments and may reduce urine flow therein which, in turn, can decrease the volume of urine that may be temporarily stored in the porous material 114. Decreasing the volume of urine that may be temporarily stored in the porous material 114 may increase the likelihood that the urine can leak therefrom.

The porous support material 126 may be selected to exhibit a surface density of between about 150 g/m² to about 800 g/m², about 150 g/m² to about 200 g/m², about 200 g/m² to about 300 g/m², about 300 g/m² to about 400 g/m² , about 400 g/m² to about 500 g/m², about 500 g/m² to about 600 g/m², about 600 g/m² to about 700 g/m², or about 700 g/m² to about 800 g/m². The surface density of the porous support material 126 is a function of the density and thickness of the porous support material 126. As such, the surface density of the porous support material 126 may be selected for any of the same reasons as the thickness of the porous support material 126.

In some embodiments, the porous material 114 can further include a supporting layer 130. The supporting layer 130 can be positioned between the porous support material 126 and the hydrophilic cover sheet 128. The supporting layer 130 can be configured to form a pathway for bodily fluids to flow. In an embodiment, the supporting layer 130 is formed from a plurality of fibers, such as a plurality of microfilaments. In an example, the plurality of fibers may be aligned in a first direction, wherein the first direction generally extends from the porous support material 126 to the hydrophilic cover sheet 128 (e.g., aligned generally perpendicularly to the longitudinal axis β-β). Aligning the fibers in the first direction allows the supporting layer 130 to more securely attach the porous support material 126 and the hydrophilic cover sheet 128 together. Further, the urine may be slightly more likely to flow in a direction that is parallel to the fibers. As such, aligning the fibers in the second direction may pull the urine through the supporting layer 130 quicker than if the fibers where otherwise oriented which causes the urine to flow in a greater percentage of the supporting layer 130 than if the urine was aligned in another direction. Causing the urine to flow in a greater percentage of the supporting layer 130 may cause a greater volume of the urine to flow through the porous material 114 at any given time and decrease the likelihood that the urine leaks from the porous material 114.

The supporting layer 130 may be formed from either a hydrophilic and/or hydrophobic material. In an example, the supporting layer 130 may be formed from a hydrophilic material. As previously discussed, porous materials of conventional fluid collection assemblies may not be formed of hydrophilic materials since such materials generally retain bodily fluids. However, when the porous support material 126 exhibits a hydrophobicity, the hydrophobicity limits the urine that may be retained by the supporting layer 130. When the supporting layer 130 is hydrophilic, the supporting layer 130 may generally exhibit a hydrophilicity that is less than the hydrophilic cover sheet 128. In an example, the supporting layer 130 may be formed from a hydrophobic material exhibiting any of the hydrophobicities disclosed herein. In such an example, the supporting layer 130 may exhibit a hydrophobicity that is less than the porous support material 126 to promote the bodily fluids flowing from the hydrophilic cover sheet 128 to the supporting layer 130. In some embodiments, the supporting layer 130 may be omitted from the porous material 114 and not included in the urine collection assembly 100.

The porous support material 126, the hydrophilic cover sheet 128, and/or the supporting layer 130 may be formed from any suitable material. In an example, at least one of the porous support material 126, the hydrophilic cover sheet 128, or the supporting layer 130 may include a base material that is coated with a material. In such an example, the coating material may exhibit a hydrophilicity or hydrophobicity that is different than the base material. In an example, at least one of the porous support material 126, the hydrophilic cover sheet 128, and the supporting layer 130 may be formed from at least one material that is treated to change a hydrophilicity or hydrophobicity thereof.

In some examples, the porous material 114 disclosed herein may include one or more additional layers. The porous material 114 and/or any of the components thereof may be composed to wick the urine away from the opening 104, thereby preventing the urine from escaping the chamber 112. The permeable properties referred to herein may be wicking, capillary action, diffusion, or other similar properties or processes, and are referred to herein as “permeable” and/or “wicking.” Such “wicking” and/or “permeable” properties may not include absorption of the bodily fluids into at least a portion of the porous material 114. Put another way, substantially no absorption or solubility of the bodily fluids into the material may take place after the material is exposed to the urine and removed from the urine for a time. While no absorption or solubility is desired, the term “substantially no absorption” may allow for nominal amounts of absorption and/or solubility of urine and/or bodily fluids into the porous material 114 (e.g., absorbency), such as less than about 30 wt % of the dry weight of the porous material 114, less than about 20 wt %, less than about 10 wt %, less than about 7 wt %, less than about 5 wt %, less than about 3 wt %, less than about 2 wt %, less than about 1 wt %, or less than about 0.5 wt % of the dry weight of the porous material 114. The porous material 114 may also wick the bodily fluids generally towards an interior of the chamber 112, as discussed in more detail below. In an embodiment, the porous material 114 may include at least one absorbent or adsorbent material.

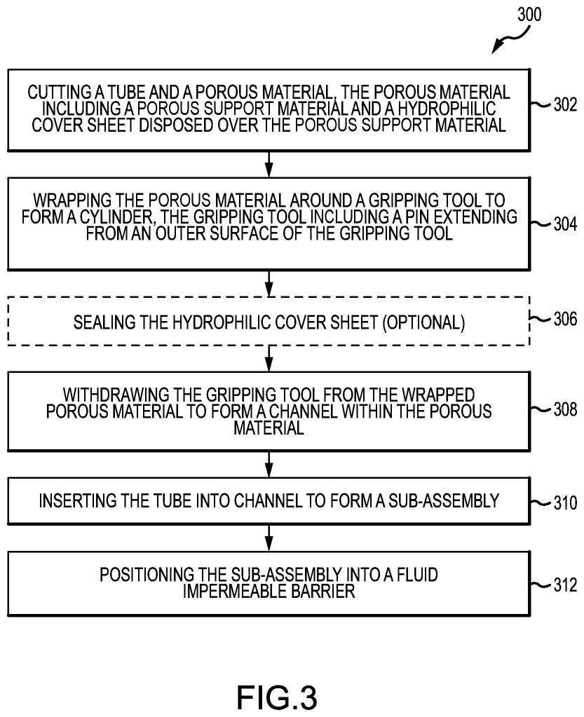

FIG. 1C is a cross-sectional schematic of the urine collection assembly shown in FIG. 3A taken along plane a-a. The porous material 114 is shown wrapped in a cylindrical shape around the tube 116 and sealed. In some embodiments, the porous material 114 may be sealed with an adhesive seal 132 and disposed within the fluid impermeable barrier 102. In some embodiments, the seal may be formed by an ultrasonic weld. In some embodiments, the material of the seal 132 may be formed from polymer adhesives such as epoxies, acrylates, or silicone materials. The adhesive seal 132 may include a curing temperature below about 200 °C. The sealing material may exhibit a coefficient of thermal expansion below about 50 ppm per degree C. In some examples, the seal 132 is moisture proof or moisture resistant. The seal 132 is provided for holding the side edges of the porous material 114 together. The seal 132 may be applied to the porous support material 126, the hydrophilic cover sheet 128, and/or the supporting layer 130. Generally, the seal 132 is applied to all components of the porous material 114. In some examples, edges of the porous material 114 may be overlapped and the seal 132 can be applied to a top and bottom surface of the edges as required to secure the porous material 114 into shape.

FIG. 1D is a side elevation view of a sub-assembly 134 of a urine collection assembly 100, according to an embodiment. The sub-assembly 134 may include the tube 116 wrapped with the porous material 114 in a cylindrical shape around the tube 116. The sub-assembly 134 includes at least the porous support material 126 and the hydrophilic cover sheet 128 disposed over the porous support material 126. The seal 132 may be applied the entire length of the sub-assembly 134. The adhesive materials of the seal 132 can be applied onto opposed surfaces of the edges of the porous material 114 and may be cured by exposure to radiant or entrained heat, radiation, air or other suitable curing medium. The seal 132 can be formed by a nontoxic adhesive, by plastic welding, by ultrasonic welding, or by a material including, but not limited to polymeric or non-woven tape. The seal 132 can be applied without damaging or affecting the properties of the components of the porous material 114. The seal 132 can include multiple layers. In some examples the layers can include different drying and/or adhesive properties. In some examples, a first adhesive layer may be fast curing and another adhesive layer may be waterproof or resistant to fluids. In some embodiments, the side edges of the porous material 114 can be cut in an interlocking pattern (e.g., zig-zag or inter-digitations) to enhance the strength of the seal 132. The pattern may be a repeating pattern or a non-repeating pattern.

FIG. 2 is a block diagram of a urine collection system 200 for urine collection assembly 202, according to an embodiment. The urine collection system 200 includes a urine collection assembly 202, a urine storage container 206, and a vacuum source 208. The urine collection assembly 202 may be the same or substantially similar to any of the fluid collection assemblies disclosed herein. The urine collection assembly 202, the urine storage container 206, and the vacuum source 208 may be fluidly coupled to each other via one or more tubes 204. For example, urine collection assembly 202 may be operably coupled to one or more of the urine storage container 206 or the vacuum source 208 via the tube 204. The urine collected in the urine collection assembly 202 may be removed from the urine collection assembly 202 via the tube 204 which may protrude into the urine collection assembly 202. For example, an inlet of the tube 204 may extend into the urine collection assembly 202, such as to a reservoir therein. The outlet of the tube 204 may extend into the urine collection assembly 202 or the vacuum source 208. Suction force may be introduced into the chamber of the urine collection assembly 202 via the inlet of the tube 204 responsive to suction (e.g., vacuum) force applied at the outlet of the tube 204.

The suction force may be applied to the outlet of the tube 204 by the vacuum source 208 either directly or indirectly. The suction force may be applied indirectly via the urine storage container 206. For example, the outlet of the tube 204 may be disposed within the urine storage container 206 and an additional tube 204 may extend from the urine storage container 206 to the vacuum source 208. Accordingly, the vacuum source 208 may apply suction to the urine collection assembly 202 via the urine storage container 206. The suction force may be applied directly via the vacuum source 208. For example, the outlet of the tube 204 may be disposed within the vacuum source 208. An additional tube 204 may extend from the vacuum source 208 to a point outside of the urine collection assembly 202, such as to the urine storage container 206. In such examples, the vacuum source 208 may be disposed between the urine collection assembly 202 and the urine storage container 206.

The urine storage container 206 may be sized and shaped to retain bodily fluids therein. The urine storage container 206 may include a bag (e.g., drainage bag), a bottle or cup (e.g., collection jar), or any other enclosed container for storing bodily fluids such as urine. In some examples, the tube 204 may extend from the urine collection assembly 202 and couple to the urine storage container 206 at a first point therein. An additional tube 204 may be coupled to the urine storage container 206 at a second point thereon and may extend and attach to the vacuum source 208. Accordingly, a vacuum (e.g., suction) may be drawn through urine collection assembly 202 via the urine storage container 206. Bodily fluids, such as urine, may be drained from the urine collection assembly 202 using the vacuum source 208. In some examples, the urine storage container 206 can include a scale or gage to measure the volume of urine collected.

The vacuum source 208 may include one or more of a manual vacuum pump, and electric vacuum pump, a diaphragm pump, a centrifugal pump, a displacement pump, a magnetically driven pump, a peristaltic pump, or any pump configured to produce a vacuum. The vacuum source 208 may provide a vacuum or suction to remove bodily fluids from the urine collection assembly 202. In some examples, the vacuum source 208 may be powered by one or more of a power cord (e.g., connected to a power socket), one or more batteries, or even manual power (e.g., a hand operated vacuum pump). The vacuum source 208 disclosed herein may further include one or more of a switch, a button, a plug, a remote, or any other device suitable to activate the vacuum source 208.

A method of forming a urine collection system, such as the urine collection system 200, can include forming and/or manufacturing the urine collection assembly 202 and fluidly connecting the urine collection assembly 202 with the urine storage container 206 and vacuum source 208 such that, when urine is present in the chamber, a suction provided from the vacuum source 208 to the chamber 118 of the urine collection assembly 202 removes the urine from the urine collection assembly 202.

FIG. 3 is a flow diagram of a method 300 of forming a urine collection assembly (e.g., urine collection assembly 100), according to an embodiment. In some examples, the method may include an act 302 of cutting a tube and a porous material. In some embodiments, the tube and the porous material may be die cut. In other embodiments, the tube and/or the porous material may be laser cut. Laser cutting works by directing the output of a high-power laser. A focused laser beam can be directed at the porous material and/or the tube, which then either melts, burns, vaporizes away, or is blown away by a jet of gas, leaving an edge with a surface finish. A die is a pre-shaped tool that works in conjunction with a press to manipulate the material into the desired size and shape. In some embodiments, the porous material is die cut to facilitate rapid manufacturing/assembly of the urine collection assembly. Die cutting is a process in which you use a machine to mass-produce cut-out shapes. The die cutter can be a manual or industrial design. In some examples, the entire process of cutting the tube and/or porous material is automated so more shapes can be produced at a faster rate. The tube and the porous material may be cut simultaneously in some embodiments. In other embodiments, the tube and porous material may be cut separately. The porous material can include at least a porous support material and a hydrophilic cover sheet disposed over the porous support material. In some examples, the porous material can further include a supporting layer. The supporting layer can be positioned between the porous support material and the hydrophilic cover sheet.

The method also may include an act 304 of wrapping the porous material that includes at least the porous support material and the hydrophilic cover around a gripping tool to form a cylinder. The gripping tool can include a pin extending from an outer surface of the gripping tool to extend into the porous material and form the porous material into a cylinder. The method also may include an act 306 of sealing the hydrophilic cover sheet. Act 306 can be optional if sealing is required or preferred prior to assembling the urine collection assembly. In some embodiments, the hydrophilic cover sheet may be sealed with an adhesive tape or glue to retain the porous material in a cylinder shape. In some examples, the adhesive may be pre-applied prior to wrapping the porous material around the gripping tool. The hydrophilic cover sheet is configured to extend across the opening of the fluid impermeable barrier of the urine collection assembly and the hydrophilic cover sheet is configured to draw urine into the urine collection assembly.

The method also may include an act 308 of withdrawing the gripping tool from the wrapped porous support material to form a channel within the porous support material. The method 300 further includes an act 310 of inserting a tube into the channel to form a sub-assembly. The sub-assembly includes the tube wrapped with the porous material in a cylindrical shape around the tube. The porous material includes the porous support material and the hydrophilic cover sheet disposed over the porous support material. The hydrophilic cover sheet may be sealed. The method also may include an act 312 of positioning the sub-assembly into a fluid impermeable barrier. The fluid impermeable barrier can include a chamber, at least one opening, and a fluid outlet and the sub-assembly is configured to be disposed in the chamber and the tube extends through the fluid outlet In some examples, the sub-assembly may be formed with a channel therein and the tube can be inserted after the porous material is disposed in the fluid impermeable barrier. The sub-assembly may be inserted into the fluid impermeable barrier through the opening. In some examples, the tube can be inserted into the opening and through a fluid outlet in the fluid impermeable barrier. In other examples, the tube can be inserted into the channel formed in the sub-assembly after being inserted through the fluid outlet. In some examples, the assembly of the urine collection assembly and/or the sub-assembly may be automated.

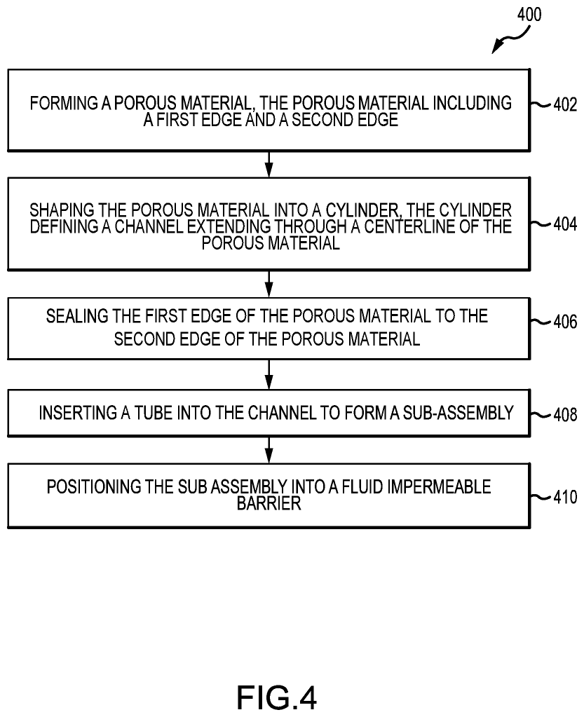

FIG. 4 is a flow diagram of a method 400 of forming a urine collection assembly (e.g., urine collection assembly 100), according to an embodiment. In some examples, the method may include an act 402 of forming a porous material including a first edge and a second edge. The second edge may be opposite the first edge. In other words, the porous material can be assembled of a porous support material and a hydrophilic cover sheet coupled to the porous material in a sheet. The porous material sheet can include a first lateral edge and a second lateral edge opposite the first lateral edge. The sheet can include other edges, as the sheet may be cut into any suitable shape including a square or rectangle. Act 402 of forming the porous material may also include coupling the porous support material and the hydrophilic cover sheet such that the hydrophilic cover sheet is disposed over the porous support material.

The method 400 also may include an act 404 of shaping the porous material into a cylinder, the cylinder defining a channel extending through a centerline of the porous material. In some examples, the porous material can be shaped by wrapping the porous material around a gripping tool to form a cylinder. The gripping tool can include a pin extending from an outer surface of the gripping tool to extend into the porous material and rolling the gripping tool to form the porous material into a cylinder. The gripping tool can then be removed to form the channel.

The method also may include an act 406 of sealing the first edge of the porous material to the second edge of the porous material. In some examples, the hydrophilic cover sheet can be sealed with an adhesive tape or glue. In some embodiments, sealing the first edge of the porous material to the second edge of the porous material can include applying a polymer adhesive to at least the first edge of the porous material. In some embodiments, the seal can extend from an outer surface of the first edge of the porous material to an inner surface of the first edge of the porous material. In other words, the seal can be applied along an entire intersection plane of the porous material. The adhesive can be configured to hold the porous material in a cylindrical shape. In some embodiments, sealing the first edge of the porous material to the second edge of the porous material can include ultrasonic welding the first edge of the porous material to the second edge of the porous material.

The method 400 further includes an act 408 of inserting a tube into the channel to form a sub-assembly. The sub-assembly includes the tube wrapped with the porous material sealed in a cylindrical shape around the tube. The method also may include an act 410 of positioning the sub-assembly into a fluid impermeable barrier. The fluid impermeable barrier can include a chamber, at least one opening, and a fluid outlet and the sub-assembly is configured to be disposed in the chamber and the tube extends through the fluid outlet.

In some examples, the sub-assembly may be formed with a channel therein and the tube can be inserted after the porous material is disposed in the fluid impermeable barrier. The sub-assembly may be inserted into the fluid impermeable barrier through the opening. In some examples, the tube can be inserted into the opening and through a fluid outlet in the fluid impermeable barrier. In other examples, the tube can be inserted into the channel formed in the sub-assembly after being inserted through the fluid outlet. In some examples, the assembly of the urine collection assembly and/or the sub-assembly may be automated.

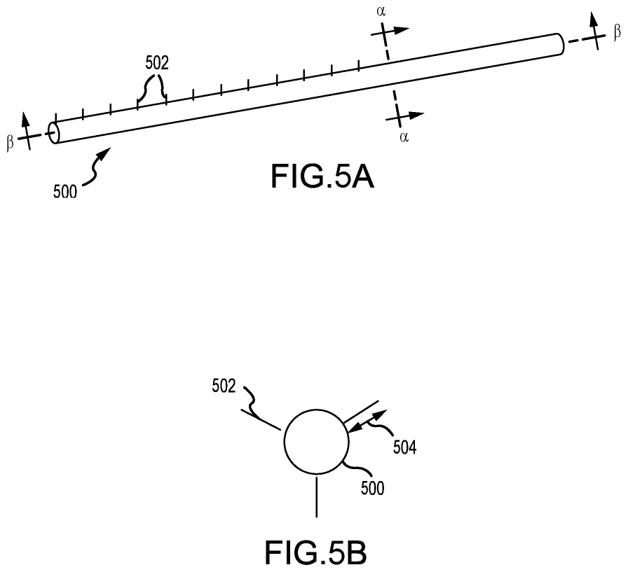

FIG. 5A is an isometric view of a gripping tool 500 including a series of pins 502, according to an embodiment and FIG. 5B a cross-sectional schematic of a gripping tool 500, according to an embodiment, taken along plane a-a, shown in FIG. 5A. In some examples, the gripping tool 500 includes a pin 502 extending from an outer surface. The pin 502 is configured to insert into the porous material, through the hydrophilic cover sheet and into the porous support material to facilitate the wrapping the porous material into a cylindrical shape. In assembling the urine collection assembly, the pin 502 of the gripping tool 500 can be attached to the porous material and the porous material wound around the gripping tool 500 to form the cylinder shape. In some examples, the porous material is wrapped in a single layer and sealed with an adhesive. The gripping tool 500 can then be removed from the porous material leaving the porous material in a cylindrical shape having a channel therein, the channel large enough for a tube (e.g., tube 116) to be inserted.

In some embodiments, the gripping tool 500 can be formed from at least one of a metal, a plastic, or a wood. The gripping tool 500 can be rigid to control the wrapping of the porous support material and the hydrophilic cover around the gripping tool 500 to form a cylinder. The pin 502 can include a series of pins 502 extending from the gripping tool 500 at least the length of the cut porous material. The gripping tool 500 can further include a portion that doesn't include pin(s) 502 thereon, to either attach to a machine or act as a handle for the gripping tool 500 during the method of forming the sub-assembly.

In some examples, as shown in FIG. 5B, the pin(s) 502 can include a series of pins disposed about the circumference of the gripping tool. The pin(s) 502 can be formed from at least one of a metal, a plastic, or a wood. The pin(s) can be formed from rigid material, strong enough to be inserted through the porous material. In some examples, the pin(s) 502 can be flexible to aid in withdrawing the gripping tool 500 from the sub-assembly. In some examples, the pin(s) 502 can be configured to bend as the gripping tool 500 is withdrawn. In other examples, the pin(s) 502 can be retracted into the interior of the gripping tool 500 to aid withdrawal. The pin(s) 502 can extends from the outer surface of the tube enough to penetrate the hydrophilic cover sheet and extend into the porous support material. In some embodiments, the pin(s) 502 can exhibit a length 504 between about 5 mm and about 10 mm.

While various aspects and embodiments have been disclosed herein, other aspects and embodiments are contemplated. The various aspects and embodiments disclosed herein are for purposes of illustration and are not intended to be limiting.

Terms of degree (e.g., “about,” “substantially,” “generally,” etc.) indicate structurally or functionally insignificant variations. In an example, when the term of degree is included with a term indicating quantity, the term of degree is interpreted to mean ±10%, ±5%, or ±2% of the term indicating quantity. In an example, when the term of degree is used to modify a shape, the term of degree indicates that the shape being modified by the term of degree has the appearance of the disclosed shape. For instance, the term of degree may be used to indicate that the shape may have rounded corners instead of sharp corners, curved edges instead of straight edges, one or more protrusions extending therefrom, is oblong, is the same as the disclosed shape, etc.

1. A method of forming a urine collection assembly, the method comprising:
cutting a tube and a porous material, the porous material including:
a first edge and a second edge opposite the first edge;
a porous support material; and
a hydrophilic cover sheet disposed over the porous support material;
wrapping the porous material around a gripping tool to form a cylinder, the gripping tool including a pin extending from an outer surface of the gripping tool;
withdrawing the gripping tool from the wrapped porous material to form a channel within the porous material;
sealing the first edge of the porous material to the second edge of the porous material;
inserting the tube into the channel to form a sub-assembly; and
positioning the sub-assembly into a fluid impermeable barrier.

2. The method of claim 1, further including sealing the hydrophilic cover sheet.

3. The method of claim 2, wherein sealing the hydrophilic cover sheet includes sealing the hydrophilic cover sheet with an adhesive tape or glue.

4. The method of claim 1, wherein cutting the tube and the porous material includes die cutting or laser cutting.

5. The method of claim 1, wherein the tube and the porous material are cut simultaneously.

6. The method of claim 1, wherein the gripping tool is formed from at least one of a metal, a plastic, or a wood.

7. The method of claim 1, wherein the pin extends from the outer surface of the tube from about 5 mm to about 10 mm.

8. The method of claim 1, wherein the pin includes a series of pins extending from the gripping tool, the series of pins extending along the gripping tool at least a length of the cut porous material.

9. The method of claim 1, wherein the pin includes a series of pins disposed about a circumference of the gripping tool.

10. The method of claim 1, wherein the pin is formed from at least one of a metal, a plastic, or a wood.

11. The method of claim 1, wherein the fluid impermeable barrier includes a chamber, at least one opening, and a fluid outlet and the sub-assembly is configured to be disposed in the chamber and the tube extends through the fluid outlet.

12. The method of claim 11, wherein the hydrophilic cover sheet extends across the at least one opening of the fluid impermeable barrier and the hydrophilic cover sheet is configured to draw urine into the urine collection assembly and toward the tube.

13. A method of forming a urine collection assembly, the method comprising:
forming a porous material, the porous material including a first edge and a second edge opposite the first edge;
shaping the porous material into a cylinder, the cylinder defining a channel extending through a centerline of the porous material;
sealing the first edge of the porous material to the second edge of the porous material;
inserting a tube into the channel to form a sub-assembly; and
positioning the sub-assembly into a fluid impermeable barrier.

14. The method of claim 13, wherein forming the porous material includes:
coupling a porous support material to a hydrophilic cover sheet, such that the hydrophilic cover sheet is disposed over the porous support material.

15. The method of claim 13, wherein sealing the first edge of the porous material to the second edge of the porous material includes applying a polymer adhesive to at least the first edge of the porous material.

16. The method of claim 13, wherein sealing the first edge of the porous material to the second edge of the porous material includes ultrasonic welding the first edge of the porous material to the second edge of the porous material.

17. A urine collection assembly, comprising:
a fluid impermeable barrier at least defining a chamber, at least one opening, and a fluid outlet;
a porous material exhibiting a cylindrical shape disposed in the chamber, the porous material including:
a porous support material;
a hydrophilic cover sheet disposed over the porous support material; and
a seal applied along an entire intersection plane of the porous material, the seal configured to hold the porous material in a cylindrical shape; and
a tube, wherein the tube is disposed within the porous material and in fluid communication with the fluid outlet.

18. The urine collection assembly of claim 17, wherein the hydrophilic cover sheet exhibits a thickness that is about 400 μm or less.

19. The urine collection assembly of claim 17, wherein the porous support material exhibits a thickness of between about 2 mm to about 20 mm.

20. The urine collection assembly of claim 17, wherein the porous support material includes at least one of polyester, polypropylene, polyethylene, nylon, spun nylon fibers, woven fabric, vertically woven bamboo, cotton, or cellulose.

21. The urine collection assembly of claim 17, wherein the porous support material exhibits a surface density of between about 150 g/m² to about 800 g/m².

22. The urine collection assembly of claim 17, further including a supporting layer disposed between the porous support material and the hydrophilic cover sheet.

23. The urine collection assembly of claim 17, wherein the hydrophilic cover sheet includes a non-woven structure material.

24. The urine collection assembly of claim 17, wherein the hydrophilic cover sheet exhibits a surface density of between about 10 g/m² to about 200 g/m².

25. The urine collection assembly of claim 17, wherein the seal includes an adhesive tape or glue.

26. The urine collection assembly of claim 17, wherein the seal includes an ultrasonic weld.
